SEQUENCE LISTING (22 sequences)

SEQ ID NO: 1 - DNA, 18 bp, Artificial Sequence
  Synthetic olignucleotide
agcggccagtataggtga

SEQ ID NO: 2 - DNA, 15 bp, Artificial Sequence
  Synthetic oligonucleotide
ttcttgcatctgtca

SEQ ID NO: 3 - DNA, 20 bp, Artificial Sequence
  Synthetic oligonucleotide
agcggacactctaggcgatg

SEQ ID NO: 4 - DNA, 20 bp, Artificial Sequence
  Synthetic oligonucleotide
gcggccaaggccgagatgca

SEQ ID NO: 5 - DNA, 20 bp, Artificial Sequence
  Synthetic oligonucleotide
tgcatctcggccttggccgc

SEQ ID NO: 6 - DNA, 20 bp, Artificial Sequence
  Synthetic oligonucleotide
cgctgcagatctctgacccg

SEQ ID NO: 7 - DNA, 20 bp, Artificial Sequence
  Synthetic oligonucleotide
cgggtcagagatctgcagcg

SEQ ID NO: 8 - DNA, 20 bp, Artificial Sequence
  Synthetic oligonucleotide
cccaccatggacaactaccc

SEQ ID NO: 9 - DNA, 20 bp, Artificial Sequence
  Synthetic oligonucleotide
gggtagttgtccatggtggg

SEQ ID NO: 10 - DNA, 20 bp, Artificial Sequence
  Synthetic oligonucleotide
tggaggagatgatgctgctg

SEQ ID NO: 11 - DNA, 20 bp, Artificial Sequence
  Synthetic oligonucleotide
cagcagcatcatctcctcca

SEQ ID NO: 12 - DNA, 20 bp, Artificial Sequence
  Synthetic oligonucleotide
catcacctatactggccgct

SEQ ID NO: 13 - DNA, 20 bp, Artificial Sequence
  Synthetic oligonucleotide
gcacccaacagtggcaacaa

SEQ ID NO: 14 - DNA, 20 bp, Artificial Sequence
  Synthetic oligonucleotide
gtgttgccactgttgggtgc

SEQ ID NO: 15 - DNA, 20 bp, Artificial Sequence
  Synthetic oligonucleotide
ccagaccagaagcccttcca

SEQ ID NO: 16 - DNA, 20 bp, Artificial Sequence
  Synthetic oligonucleotide
tggaagggcttctggtctgg

SEQ ID NO: 17 - DNA, 20 bp, Artificial Sequence
  Synthetic oligonucleotide
agctcatcaaacccagccgc

SEQ ID NO: 18 - DNA, 20 bp, Artificial Sequence
  Synthetic oligonucleotide
gcggctgggtttgatgagct

SEQ ID NO: 19 - DNA, 20 bp, Artificial Sequence
  Synthetic oligonucleotide
caagaaagcagacaaaagtg

SEQ ID NO: 20 - DNA, 20 bp, Artificial Sequence
  Synthetic oligonucleotide
cacttttgtctgctttcttg

SEQ ID NO: 21 - DNA, 20 bp, Artificial Sequence
  Synthetic oligonucleotide
gcccaggtcagcagcttccc

SEQ ID NO: 22 - DNA, 20 bp, Artificial Sequence
  Synthetic oligonucleotide
gggaagctgctgacctgggc

(19) United States
(12) Patent Application Publication
(10) Pub. No.: US 2005/0261226 A1
(43) Pub. Date: Nov. 24, 2005

(54) Methods and compositions for the treatment of cancer with oligonucleotides directed against Egr-1

(76) Inventor: Daniel Mercola, Rancho Santa Fe, CA (US)

Correspondence Address: DAVID B. WALLER & ASSOCIATES, 5677 OBERLIN DRIVE, SUITE 214, SAN DIEGO, CA, 92121, US

(21) Appl. No.: 11/082,731

(22) Filed: Mar. 17, 2005

Related U.S. Application Data
(60) Provisional application No. 60/554,586, filed on Mar. 19, 2004.

(51) Int. Cl. C12Q 1/68; C07H 21/02; A61K 48/00

(52) U.S. Cl. 514/44; 435/6; 536/23.1

(57) ABSTRACT

Oligonuclotides, compositions and methods for modulating the over-expression of EGR-1 in cancer cells. The compositions comprise antisense compounds, particularly antisense oligonucleotides, targeted to nucleic acids encoding Egr-1. Methods include using these compounds for modulation of Egr-1 expression in cancer cells in which Egr-1 is over-expressed for the treatment of cancer.

### TECHNICAL FIELD

The present invention relates generally to the field of molecular biology, in particular oligonucleotide compositions for the treatment of diseases in which Egr-1 expression levels are elevated as for example in prostate cancer.

### BACKGROUND OF THE INVENTION

Prostate cancer is the most common malignancy in men and a frequent cause of cancer death. The mortality of this disease is due to metastasis to the bone and lymph nodes. Prostate cancer progression is thought to proceed from multiple defined steps through prostatic intra-epithelial neoplasia (PIN), invasive cancer, and progression to androgen-independent and refractory terminal phase. A large fraction of early onset, and up to 5-10% of all prostate cancer patients, may have an inherited germline mutation that has facilitated the onset of carcinogenesis. However, in the majority of cases, no inherited gene defects are involved, and cancer arises as a result of a series of acquired somatic genetic changes affecting many genes on several chromosomes. Although the molecular mechanism of prostate cancer progression remains largely unknown, a few genes such as E-cadherin, α-catenin, TGF-β and insulin-like growth factors I and II (IGFs), have been shown to be aberrantly expressed and are markers of prostate cancer.

The type of treatment selected for prostate cancer is dependent upon the stage of the disease at the time of initiating treatment. Stage I or T1 is indicated when palpation of the area does not identify an abnormality or tumor. Stage 2 or T2 is indicated when an abnormality or tumor is identified by palpation of the area and the tumor or abnormality is localized to the prostate. Stage 3 or T3 is indicated when the tumor has grown through the prostate capsule and perhaps into the seminal vesicles. T4, another level of stage 3, is indicated when a tumor has grown into nearby muscles and organs. Stage 4 is indicated when the tumor has metastasized to regional lymph nodes or more distant parts of the body. Under Stage 1 or 2 a patient may elect watchful waiting, surgery or radiation therapy. At stage 3 or 4 hormonal therapy is the treatment of choice.

Currently there are six recognized treatments for prostate cancer; watchful waiting, surgery, radiation therapy, chemotherapy, hormonal therapy and cryosurgery. Watchful waiting is based on the premise that cases of localized prostate cancer may advance so slowly that they are unlikely to cause problems over the lifetime of the patient. Unfortunately, watchful waiting has the disadvantage of decreasing the chance to control the disease before it spreads or may result in postponement of treatment to an age when it may be more difficult to tolerate or recover from another treatment type such as surgery.

In the early 1990's thirty percent of prostate cancer patients were treated with surgery. Radical prostatectomy removes the entire prostate gland along with seminal vesicles both ampullae and other surrounding tissues. The section of the urethra that runs through the prostate is cut away often times removing a portion of the sphincter muscle that controls the flow of urine. A prostatectomy carries the risk of serious long-term complications notably incontinence both urinary and stool depending on the type of surgical operation utilized to remove the prostate and sexual impotence.

When prostate cancer is localized, radiation therapy serves as an alternative to surgery. Radiation therapy uses high-energy X-rays emitted from an X-ray machine or by radioactive particles implanted in the prostate. The disadvantage to radiation therapy is possible damage to healthy tissues in the region of the cancer such as the rectum, bladder and intestines. In addition, two-thirds of patients reported problems with urinary incontinence and forty to fifty percent of men became impotent.

Brachytherapy or implantation of radioactive particles in the prostate gland reduces the chance of damage to healthy tissue focusing a majority of the therapeutic effects against the cancer. However, this treatment is not well suited for large advanced tumors and may cause sexual impotence.

Conformal radiation therapy is a sophisticated three-dimensional radiation treatment using computer software to conform or shape the distribution of the radiation beams to the three-dimensional shape of the diseased prostate generally sparing damage to normal tissue in the vacinity of treatment. Unfortunately, this treatment has the disadvantage of potential urinary incontinence and impotence.

Hormonal therapy combats prostate cancer by eliminating the supply of male hormones, also known as androgens such as testosterone, that encourage prostate cancer cell growth. This therapy has the advantage of treating cancer that has spread beyond the prostate gland, which are often times beyond the reach of localized treatments such as surgery and radiation therapy. Hormonal control can be achieved by removal of the testicles, surgical castration, or by administration of drugs, medical castration. Unfortunately, both castration methods can cause hot flashes, impotence and loss of interest in sex. Medical castration has the added disadvantage of causing breast enlargement and potential cardiovascular problem such as heart attack and stroke.

Cryosurgery uses liquid nitrogen to freeze and kill prostate cancer cells. Unfortunately, the overlying nerve bundles usually freeze so most men become impotent.

Chemotherapy which kills fast growing cells has not proven particularly effective against slow growing prostate cancer cells and is therefore not a treatment of choice under these conditions.

Consequently, there is a need in the industry for a medical composition that reduces the severity of prostate cancer without causing damage to surrounding healthy tissues, urinary incontinence, stool incontinence, impotence, breast enlargement or cardiovascular problems.

### SUMMARY OF THE INVENTION

The oligonucleotides of the present invention may be used to modulate the expression of EGR-1 in cancer cells in which EGR-1 is overexpressed due to the presence of the disease. In one aspect of the invention an oligonucleotide of up to 30 bases in length is provided comprising at least an 8 nucleobase portion of the sequence 5′-AGC GGC CAG TAT AGG TGA-3′ (SEQ ID NO.1). The oligonucleotide is an antisense oligonucleotide or iRNA.

In one embodiment the oligonucleotide or iRNA may comprise at least one modified internucleosidyl linkage. At least one modified internucleosidyl linkage may be a phosphorothioate internucleosidyl linkage, a methylphosphonate internucleosidyl linkage, or a phosphodiester internucleosidyl linkage. Alternatively, the modified oligonucleotide or iRNA may comprise at least one methylphosphonate and at least one phosphodiester analog, at least one methylphosphonate and at least one phosphorothioate analog, at least one phosphorothioate and at least one phosphodiester analog or at least one methylphosphonate, at least one phosphorothioate and at least one phosphodiester analog.

In another embodiment the oligonucleotide or iRNA may comprise at least one modified sugar moiety and or at least one modified nucleobase moiety. In a preferred embodiment the oligonucleotide is a chimeric oligonucleotide.

In another aspect of the invention a method for the treatment of cancer cells wherein Egr-1 is over-expressed as a result of the cancer is provided comprising the steps of administering an oligonucleotide or iRNA described above to an animal having cancer-cells in which Egr-1 is over-expressed for a length of time and until Egr-1 expression is reduced, the proliferation of the cancer cells is decreased, or apoptosis of the cancer cells is increased. In one embodiment of this aspect of the invention the treatment method may further result in reduced expression of transforming growth factor beta-1 or interleukin-6, Cyclin D2 and G-alpha-12 in the cancer cells or in increased expression of Cyclin G2 and p19^{ink4d}.

In yet another embodiment a method of interfering with the growth of cancer cells is provided wherein the cancer cells over-express the Egr-1 gene, comprising the steps of introducing an oligonucleotide described above to cancer cells and contacting the cancer cells with an amount of at least one chemotherapeutic agent sufficient to kill a greater portion of the cancer cells than would have been killed by the same amount of chemotherapeutic agent in the absence of said oligonucleotide.

In a preferred embodiment of the present invention the cancer cells are prostate cancer cells. In other embodiments a composition is provided comprising the oligonucleotide or iRNA and a pharmaceutically acceptable carrier, a vector comprising the antisense oligonucleotide sequence, a plasmid comprising the vector and a cell comprising the antisense oligonucleotide or iRNA.

In another aspect of the invention a kit comprising the antisense oligonucleotide, an iRNA, a vector comprising the antisense oligonucleotide, or a cell comprising the antisense oligonucleotide or iRNA of the present invention and a chemotherapeutic agent is provided.

### BRIEF DESCRIPTION OF THE DRAWINGS

FIG. 1: Inhibition of Egr-1 expression by E5 antisense oligonucleotide. (A) TRAMP C2 cells were transfected with the control oligonucleotide (ctl), the antisense oligonucleotide (AS) or carrier alone (M) for 4 h. After 24 h the cells were lysed and samples were analyzed by western-blotting with antibodies to Egr-1. Membranes were reprobed successively with antibodies to Egr-3, Egr-2, WT-1 and β-actin as internal control. (B) Proteins were extracted each day for 6 days following AS (C2-AS) and ctl (C2-ctl) transfection. Samples were analyzed by western blotting with antibody to Egr-1, and antibody to β-actin to control for protein loading. (C) a table of antisense oligonucleotide sequences complimentary to mouse (Accession NM 007913) at positions identical in human;

FIG. 2: Effect of Egr-1 inhibition on proliferation. (A) Proliferation assay. TRAMP C2 cells were transfected with ctl (C2-ctl) or AS (C2-AS) antisense oligonucleotide and submitted to proliferation assay for 7 days. Each day from day 0 (D0) to day 6 (D6), the number of cells of C2-ctl (line) and C2-AS (dashed line) was counted and plotted as the mean of three separate experiments, (B) Colony forming assay. TRAMP C2 cells were transfected with 0.1 μM ctl or AS antisense oligonucleotide for 4 h. After 16 h, 200 cells were placed in each well of 6-well plates in RPMI medium containing. 0.1 μM antisense oligonucleotides. After 8 days, the colonies were stained with 2% crystal violet;

FIG. 3: Time course of mRNA expression. Cyclin D2, G-alpha-12 protein, cyclin G2 and p19^{ink4d} mRNA expression, were determined by one step real-time RT-PCR. Expression levels of each gene were normalized to the level of glyceraldehyde-3-phosphate dehydrogenase expression and the ratio between each day versus day 0 was calculated as fold induction. All reactions were performed in duplicate from two different samples corresponding to TRAMP C2 cells transfected with ctl (black boxes) and AS (grey boxes) antisense oligonucleotide;

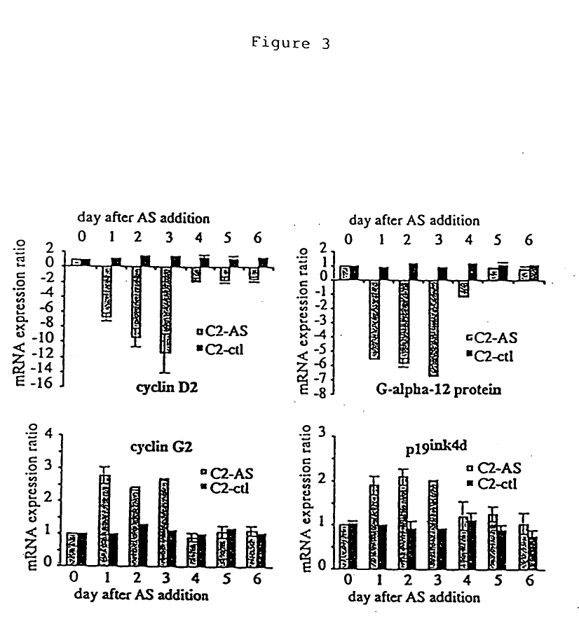

FIG. 4: Time course of protein expression. (A) Time course regulation of cyclin D2 and p19^{ink4d} protein expression. Protein extracts from C2-ctl and C2-AS cells were analyzed as described in FIGS. 1A and B by western-blotting with antibodies to Egr-1, cyclin D2, p19^{ink4d} using β-actin as a loading control. (B) Cyclin D2, G-alpha-12 protein, and p19^{ink4d} mRNA expression, were determined by one step real-time RT-PCR in mouse C2 prostate cells. Expression levels of each gene were normalized to the level of glyceraldehyde-3-phosphate dehydrogenase expression and the ratio between AS condition versus ctl oligonucleotide was calculated as fold induction. (C) Protein extracts from DU145 (lanes 1, 2, 3, 6), 267B (lane 4), P69 (lane 5) cells were analyzed by western-blotting with antibodies to Egr-1, cyclin D2 and β-actin;

FIG. 5: Inhibition of Egr-1 expression increases sensitivity to apoptotic stimuli. (A) C2-ctl and C2-AS were exposed or not to ultraviolet-C radiation (UVC 40 Jm⁻²). One and two days later dead cells were determined by trypan blue staining. The blue staining dead cell count is shown as a percentage of the total cells and the absolute number of dead and alive cells is reported within the bar chart. (B) C2-ctl and C2-AS were exposed (lane 2 and 4) or not (lane 1 and 3) to ultraviolet-C radiation (UVC 40 Jm⁻²). Twenty-four hours later proteins were extracted and subjected to analysis by western blotting with antibodies to Egr-1 or CD95. β-Actin levels were used as a loading control. (C) Fas L mediated apoptosis. C2-ctl and C2-AS were treated or untreated with 100 ng/ml Fas L for 9 h and 18 h as described in example 23. Dead cells were determined by trypan blue staining and reported as described above;

FIG. 6: Is a table illustrating that Egr-1 binds directly to p19^{ink4d}, Mad, CD95 and Cyclin D2 regulatory sequences. C2 cells were transfected (B) or not (A) with AS and ctl oligonucleotides. The cells were chromatin crosslinked and then immunoprecipitated with specific Egr-1 antibody or nonimmune control antibody. The detection of each gene in the captured fragment mix was performed by PCR as described in experimental procedures. (A) The top, middle and bottom panels show respectively, PCR products from the genomic DNA input, Egr-1-specific immunoprecipitation samples and the non-immune control from untransfected TRAMP C2 cells (mock). (B) The top and bottom panels show respectively, PCR products from Egr-1-specific immunoprecipitation samples from TRAMP C2 transfected with ctl and AS oligonucleotides. (C) Cyclin D2, p19^{ink4d} and TGF-β 1 mRNA expression, were determined by one step real-time RT-PCR. Expression levels of each gene were normalized to the level of glyceraldehyde-3-phosphate dehydrogenase expression and the ratio between 5 h, 10 h, 15 h versus 0 h was calculated as fold induction;

FIG. 7: Is a table showing data from the Affymetrix analysis of genes regulated in TRAMP C2 cells that express. Egr-1 constitutively compared with antisense treated cells. For each gene, the fold induction (Affymetrix ratio), its function (gene function), any reported involvement in human prostate cancer (link with prostate cancer) and data on its regulation by Egr-1 (known as Egr-1 target gene);

FIG. 8: Comparison of Affymetrix array with real-time RT-PCR ratio for mRNA levels. Changes in the expression level of several Egr-1 target genes given in FIG. 7 were independently tested using quantitative RT-PCR analysis of RNA from C2-ctl and C2-AS treated cells. The results were normalized to GAPDH and expressed as the ratio of C2-AS over C2-ctl values. All reactions were performed in triplicate from two different experiments and the resulting standard errors are also given. Positive and negative values mean respectively up-regulation and down regulation in response to Egr-1 inhibition (positive values indicate a down-regulation by Egr-1);

FIG. 9: Screening and characterization of the antisense oligonucleotides. (A) HT1080-E9 cells were transfected with carrier alone (mock) or with 0.1, 0.2 and 0.4 μM of the indicated oligonucleotides. Cells were lysed and protein expression was detected by western analysis using antibodies to Egr-1. Equal loading was verified by reprobing the membrane with antibodies to β-actin. A representative experiment is shown. (B) Cells were transfected with carrier alone (mock), with control or with antisense oligonucleotides at a concentration of 0.4 μM. Egr-1 protein expression was analyzed by western blot. Autoradiograms were quantified using a Kodak™ DC120-Zoom digital camera and Kodak 1D-image analysis software (Eastman Kodak Company, Rochester, N.Y.). Results (means±SE of at least three separate determinations) are expressed relative to Egr-1 expression in mock-treated cells. Position of each oligonucleotide within the nucleotide sequence of the murine gene is given in correlation with the specific preparation of antisense oligonucleotide, designated E1 to E10 (in brackets). (C) Cells were transfected with 0.3 μM control oligonucleotide (EC), E5 and E6 antisense oligonucleotides or carrier alone (M). Cells were lysed 24 h and 72 h after the start of transfection. Samples were analyzed by western using antibodies to Egr-1. Membranes were reprobed with antibodies to β-actin;

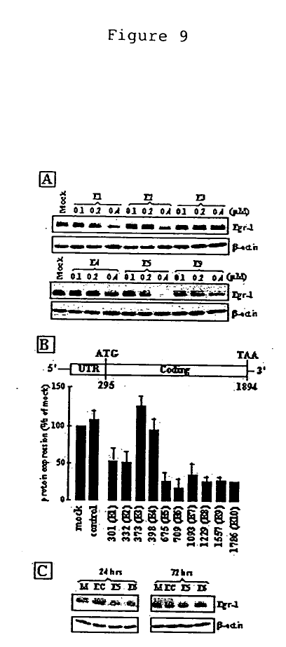

FIG. 10: Analysis of Egr-1 expression in TRAMP-C cells. (A) TRAMP-C2 cells were transfected with increasing concentrations of oligonucleotide E5, with 0.2 μM of control oligonucleotide (EC) or carrier alone (mock). After 24 h cells were lysed and samples were analyzed by western blot using antibodies to Egr-1. Membranes were reprobed with antibodies to β-actin to control for equal loading. (B) TRAMP-C cells were transfected with 0.2 μM control or E5 oligonucleotides the day before the experiment. Cells were treated with carrier alone or with TPA (50 ng/ml) for 3 h before lysis. Samples were submitted to western analysis using antibodies to Egr-1. Membranes were reprobed with antibodies to β-actin;

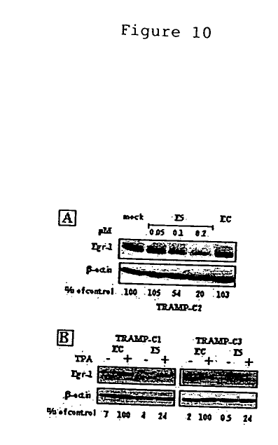

FIG. 11: Effect of antisense oligonucleotides on the expression of other Egr family members. TRAMP-C cells were transfected with carrier alone (M), control oligonucleotide (EC) or with E5 or E6 antisense oligonucleotides as indicated. (A) Protein expression was determined by western analysis. Membranes were probed successively with antibodies to Egr-3, Egr-1, Egr-2 and β-actin. A representative autoradiogram is shown. (B) mRNA expression was analyzed by RT-PCR from total RNA using probes specific for Egr-1, Egr-4 and GAPDH. PCR fragments were submitted to 2% agarose gel electrophoresis containing ethidium bromide and visualized under a UV lamp;

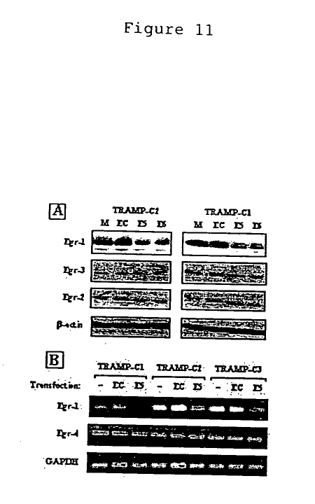

FIG. 12: Effect of antisense oligonucleotide E5 on the expression of Egr-1 target genes. (A) mRNA expression in transfected TRAMP-C cells was analyzed by RT-PCR from total RNA using probes specific for Egr-1, TGF-β1, Egr-2, PTEN or GAPDH as a control. PCR fragments were submitted to 2% agarose gel electrophoresis containing ethidium bromide and visualized under a UV lamp. (B) Protein expression in transfected TRAMP-C cells was analyzed by western blot using antibodies to Egr-1. Membranes were reprobed successively with antibodies to PTEN and β-Actin;

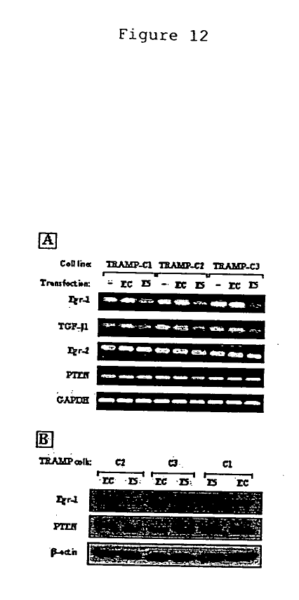

FIG. 13: Effect of the antisense oligonucleotides on cell growth and cell cycle progression. (A) TRAMP-C cells were transfected with 0.1 μM control or antisense oligonucleotide E5. They were counted as described in Examples 3 and 4 using a Coulter counter. Results represent means±SE from 3 separate experiments and are expressed relative to control oligonucleotide-treated cells at the maximum of the growth curve, i.e. at t=90 h. (B) TRAMP-C2 cells were transfected with 0.1 μM control, antisense oligonucleotide E5 or antisense oligonucleotide E6. Results are expressed relative to control oligonucleotide-treated cells at the maximum of the growth curve (t=90 h). (C) A fraction of transfected cells was lysed at t=28 h and analyzed by western blot for Egr-1 expression. Membranes were reprobed with antibodies to β-actin. A representative blot from one of the three experiments is shown. (D) TRAMP-C2 cells were transfected with control or E5 antisense oligonucleotide (0.2 μM). Forty-eight hours after transfection they were suspended by trypsin digestion and fixed in 70% ethanol. They were stained with propidium iodine as described in Example 8 and subjected to flow cytometry for DNA analysis;

FIG. 14: Effect of antisense oligonucleotide E5 on colony formation and growth in soft agar. (A) TRAMP-C cells were transfected with control or E5 antisense oligonucleotide (0.1 μM). One day later they were replated at a density of 200 cells/plate in 10% FBS-containing medium. Cells were stained with crystal violet after a week. A representative picture of colony staining is shown. (B) Colonies were counted and the actual number of colony/plate was plotted means±SE of 3 separate experiments, each performed in three to six replicate wells). (C) Transfected cells were seeded into a 0.35% agar layer on top of a 0.5% agar bottom. After 2 weeks, cells were stained with nitro-blue tetrazolium. Colonies were counted and the total colony number from each experiment was plotted (mean from 2 separate experiments, each performed in three to six replicate wells). (D) A fraction of transfected cells was lysed the day after transfection and analyzed by western blot for Egr-1 expression. Membranes were reprobed with antibodies to β-actin; and

FIG. 15: Inhibition of tumor incidence in TRAMP mice. TRAMP mice were treated with saline injections (n=7), with control scramble oligonucleotide (n=8) or with E5 antisense oligonucleotide (n=8). Animals were sacrificed after ten weeks of treatment (average age of 32 weeks). (A) Percentage of tumor incidence in the three treatment groups. (B) A tumor-bearing mouse from control oligonucleotide group (left) and a tumor-free mouse from the antisense oligonucleotide group (right). The arrow points to the tumor. (C) Tumor size for each mouse. Medians are indicated by horizontal bars.

### DETAILED DESCRIPTION OF THE INVENTION

Prior to setting forth the invention, it may be helpful to an understanding thereof to first set forth definitions of certain terms that will be used herein after. These terms will have the following meanings unless explicitly stated otherwise.

The terms “target nucleic acid” and “nucleic acid encoding EGR-1” encompass DNA encoding EGR-1, RNA (including pre mRNA and mRNA) transcribed from such DNA, and also cDNA derived from such RNA.

The term “modulation” refers either to an increase (stimulation) or a decrease (inhibition) in the expression of a gene.

The terms “start codon region” and “translation initiation codon region” refer to a portion of such a mRNA or gene that encompasses from about 25 to about 50 contiguous nucleotides in either direction (i.e., 5′ or 3′) from a translation initiation codon.

The term “hybridization” refers to hydrogen bonding, which may be Watson-Crick, Hoogsteen or reversed Hoogsteen hydrogen bonding, between complementary nucleoside or nucleotide bases. For example, adenine and thymine are complementary nucleobases, which pair through the formation of hydrogen bonds.

The term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

The term “chimeric” antisense compounds or “chimeras” refer to antisense compounds, particularly oligonucleotides, which contain two or more chemically distinct regions, each made up of at least one monomer unit, i.e., a nucleotide in the case of an oligonucleotide compound.

The term “prodrug” refers to a therapeutic agent that is prepared in an inactive form that is converted to an active form (i.e., drug) within the body or cells thereof by the action of endogenous enzymes or other chemicals and/or conditions.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

One of the over-expressed genes found in prostate cancer tissue is the transcription factor Early growth response gene 1 (Egr-1). This gene could have an important function because its expression level increases with the degree of malignancy as measured by the Gleason grade of the tumor. This seems to be specific to prostate tumor cells because in mammary and lung tumors, as well as most normal tissues, Egr-1 expression is low. Egr-1 over-expression is correlated with the loss of its co-repressor NAB2 in primary prostate carcinoma. This disruption of the balance between Egr-1 and NAB2 expression results in a high Egr-1 transcriptional activity in prostate carcinoma cells. There are at least two known mechanisms that enhance the activity of Egr-1 in human cancer cells that may act synergistically, increasing Egr-1 protein and increased Egr-1 transactivation activity. It may be important to note that the increased activity when combined with the increased protein expression Egr-1 may produce a large effect on gene regulation that causes or supports the transformed phenotype type. A recent study based on the cross breeding of Egr-1^{-/-} mice with TRAMP mice showed significantly delayed prostate tumor formation in the Egr-0.1-deficient TRAMP mouse compared to TRAMP-Egr-1^{+/+} mice. The TRAMP mouse is a well-known model of prostate cancer in which tumors progress to metastases in a window from 8 to 24 weeks of age. Although Egr-1 loss did not appear to prevent tumor initiation, Egr-1 deficiency delayed the progression of prostate tumors in these mice. Significantly, several gene products associated with aggressive prostate cancer such as TGF-β and insulin-like growth factor II have been identified as regulated by Egr-1. These observations strongly suggest that Egr-1 is involved in prostate cancer progression despite its known role as a tumor-suppressor in several other types of human cancers.

The Gleason grading system assigns a grade to each of the two largest areas of cancer in the tissue samples. Grades range from 1 to 5, with 1 being the least aggressive and 5 the most aggressive. Grade 3 tumors, for example, seldom have metastases, but metastases are common with grade 4 or grade 5. The two grades are then added together to produce a Gleason score. A score of 2 to 4 is considered low grade; 5 through 7, intermediate grade; and 8 through 10, high grade. A tumor with a low Gleason score typically grows slowly enough that it may not pose a significant threat to the patient in his lifetime.

The present invention employs oligomeric antisense compounds, particularly oligonucleotides, for use in modulating the function of nucleic acid molecules encoding EGR-1, ultimately modulating the amount of EGR-1 produced and may lead to the complete destruction of the target gene by the action of endogenous nuclear Ribonuclease-H and other endogenous endonucleases. This is accomplished by providing antisense compounds, which specifically hybridize with one or more nucleic acids encoding EGR-1. The specific hybridization of an oligomeric compound with its target nucleic acid interferes with the normal function of the nucleic acid and may lead to the destruction of the target nucleic acid by the action of ribonuclease or other endogenous endonucleases. This interference resulting in modulation of function of a target nucleic acid by compounds, which specifically hybridize to it, is generally referred to as “antisense”. The functions of DNA that may be interfered with include replication and transcription. The functions of RNA that may be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of EGR-1. In the context of the present invention, reduction in gene expression is the preferred form of modulation and mRNA is a preferred target. In particular it is desirable to reduce Egr-1 expression in disease states in which Egr-1 expression is elevated as a result of the presence of the disease for example in prostate cancer and other diseases related to the mitogenic activity of Egr-1 such as coronary heart disease, atherosclerosis and other inflammatory conditions.

It is preferred to target specific nucleic acid sequences of genes intended for expression modulation. Targeting an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding EGR-1. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation codon of the open reading frame (ORF) of the gene. Particularly preferred is the initiation codon open reading frame. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes that have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding EGR-1, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “stop codon region” and “translation termination codon region” refer to a portion of such an mRNA or gene that encompasses from about 25 to about 50 contiguous nucleotides in either direction (i.e. 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of a mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as introns, which are excised from a transcript before it is translated. The remaining (and therefore translated) regions are known as exons and are spliced together to form a continuous mRNA sequence. mRNA splice sites, i.e., intron/exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In another embodiment of the invention the inhibition of Egr-1 may be effected by the use of small interference ribonucleic acid (iRNA) (Bartel, Cell 116(2):281-97, 2004; Weiner, Mol. Cell. 12(3):535-6 Review, No abstract available. 2003). Interference RNA is the process where the introduction of double stranded RNA into a cell inhibits gene expression in a sequence dependent fashion. iRNA is seen in a number of organisms such as Drosophila, nematodes, fungi and plants, and is believed to be involved in anti-viral defense, modulation of transposon activity, and regulation of gene expression. The RNA may be an oligonucleotide RNA bound to complementary RNA to make double stranded RNA. The double stranded iRNA may be introduced into target cells or tissue by transfection, such as by lipid-mediated or lipofection. The transfected RNA combines with homologous sequences of the target transcript or mRNA leading to destruction. The process is called post-transcriptional gene silencing (Bernstein, Nature 2001 409(6818) 363-6, 2001). Post transcriptional gene silencing is believed to be due to a nuclease activity that specifically degrades exogenous transcripts homologous to transfected double-stranded RNA. This enzyme contains an essential RNA component, which may be important in directing the nuclease to the iRNA-mRNA complex.

Once one or more target sites have been identified, oligonucleotides are chosen which are sufficiently complementary to the sequence of the target, i.e., hybridize sufficiently well and with sufficient specificity, to give the desired effect.

The terms specifically hybridizable and complementary are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA causes a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

While antisense oligonucleotides are a preferred form of antisense compound, the present invention comprehends other oligomeric antisense compounds, including but not limited to oligonucleotide mimetics. The antisense compounds in accordance with this invention preferably comprise from about 8 to about 30 nucleobases. Particularly preferred are antisense oligonucleotides comprising from about 8 to about 30 nucleobases (i.e. from about 8 to about 30 linked nucleosides). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside linkages or backbone of the oligonucleotide. The natural linkage or backbone of RNA and DNA is a 3′ to 5′ phosphodiester linkage.

Backbone Constructions

Specific examples of preferred antisense compounds useful in this invention include oligonucleotides containing modified backbones or non-natural internucleoside linkages. Oligonucleotides having modified backbones include those that retain a phosphorus atom in the backbone and those that do not have a phosphorus atom in the backbone.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,6.77; 5,541,307; 5,561,225; 5,5.96,086; 5,602,240; 5,610,2.89; 5,602,240; 5,608,046; 5,610,28.9; 5,618,704; 5,623,070; 5,663,312; 5,633,360; 5,677,437; and 5,677,439, each of which is herein incorporated by reference.

Most preferred embodiments of the invention are oligonucleotides with phosphorothioate backbones and oligonucleosides with heteroatom backbones and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— (wherein the native phosphodiester backbone is represented as —O—P—O—CH₂—) of the above referenced U.S. Pat. No. 5,489,677 and the amide backbones of the above referenced U.S. Pat. No. 5,602,240. Also preferred are oligonucleotides having morpholino backbone structures of the above-referenced U.S. Pat. No. 5,034,506.

Sugar Moiety

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE.

Other preferred modifications include 2′-methoxy (2′-O—CH₃), 2′-aminopropoxy (2′-OCH₂CH₂CH₂NH₂) and 2′-fluoro (2° F.). Similar modifications may also be made at other positions on the oligonucleotide, particularly the 3′ position of the sugar on the 3′ terminal nucleotide of in 2′-5′ linked oligonucleotides and the 5′ position of 5′ terminal nucleotide. Oligonucleotides may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427, 5,591,722; 5,597,909; 5,610,300; 5,627,053; 5,639,873; 5,646,265; 5,658,873; 5,670,633; and 5,700,920, each of which is herein incorporated by reference in its entirety.

Base Moiety

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5′-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadeine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley and Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,681,941, and 5,750,692 each of which is herein incorporated by reference.

Other Moieties

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86:6553-6556), cholic acid (Manoharan et al., Bioorg. Med. Chem. Let., 1994, 4:1053-1060), a thioether, e.g., hexyl-S-tritylthiol (Manoharan et al., Ann. N.Y. Acad. Sci., 1992, 660:306-309; Manoharan et al., Bioorg, Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J., 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyl-oxycholesterol moiety (Crooke et al., J. Pharmacol. Exp. Ther., 1996, 227:923-937.

Representative U.S. patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5,109,124; 5,118,802; 5,138,045; 5,414,077, 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830, 5,112,963; 5,214,136; 5,245,136; 5,245,022; 5,254,469; 5,258,506; 5,262,536; 5,272,250; 5,292,873; 5,317,098; 5,371,241; 5,391,723; 5,416,203; 5,451,463; 5,510,475; 5,512,667; 5,514,785; 5,565,552, 5,567,810; 5,574,142; 5,858,481; 5,857,371; 5,595,726; 5,597,696; 5,599,923; 5,599,928 and 5,688,941 each of which is herein incorporated by reference.

It is not necessary for all positions in a given compound to be uniformly modified, and in fact more than one of the aforementioned modifications may be incorporated in a single compound or even at a single nucleoside within an oligonucleotide.

The present invention also includes antisense compounds, which are chimeric compounds. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides and/or modified oligonucleotides as described above. Such compounds have also been referred to in the art as hybrids or gapmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922 each of which is herein incorporated by references in its entirety.

Synthesis

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally use similar techniques to prepare oligonucleotides such as phosphorothioates and alkylated derivatives (Kurreck Eur. J. Biochem. 270(8):1628-44, 2003; Fearon et al. Ciba Found Symp. 209:19-31; discussion 31-7, 1997; Zon and Geiser Anticancer Drug Des. 6(6):539-68, 1991; Morvan et al. Anticancer Drug Des. 6(G):521-9, 1991; and Stein et al. Pharmacol Ther. 52(3):365-84, 1991).

The antisense compounds of the invention are synthesized in vitro and do not include antisense compositions of biological origin, or genetic vector constructs designed to direct the in vivo synthesis of antisense molecules.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative U.S. patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Pharmaceutically Acceptable Salts

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

In particular, prodrug versions of the oligonucleotides of the invention are prepared as SATE [(S-acetyl-2-thioethyl) phosphate] derivatives according to the methods disclosed in WO 93/24510 to Gosselin et al. or in WO 94/26764 to Imbach et al.

Pharmaceutically acceptable base addition salts are formed with metals or amines, such as alkali and alkaline earth metals or organic amines. Examples of metals-used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66:1-19). The base additional salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. A pharmaceutical additional salt includes a pharmaceutically acceptable salt of an acid form and may be organic or inorganic acid salts of amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1-5-disulfonic acid, 2- or 3-phosphoglycerate, glucose-6-phosphate, N-cyclohexysulfamic acid (with the formation of cyclamates), or with other acid organic compounds, such as ascorbic acid.

Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalene-disulfonic acid, polygalacturonic acid, and the like; and (d) salts formed from elemental anions such as chlorine, bromine, and iodine.

Administration

The present invention also includes pharmaceutical compositions and formulations, which include the antisense compounds of the invention, may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, interarterial, subcutaneous, intraperitoneal or intramuscular injection or infusion; or intracranial, e.g., intrathecal or intraventricular, administration. Oligonucleotides with at least one 2′O-methoxyethyl modification are believed to be particularly useful for oral administration.

Pharmaceutical compositions and formulations for topical administration may include transdermal patches, ointments, lotions, creams, gels, drops, suppositories, sprays, liquids and powders.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

Formulations

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances, which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

The compositions of the present invention may be prepared and formulated as emulsions. Emulsions are typically heterogeneous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301).

The application of emulsion formulations via dermatological, oral and parenteral routes and methods for their manufacture have been reviewed in the literature (Idson, supra). Emulsion formulations for oral delivery have been very widely used because of reasons of ease of formulation, efficacy from an absorption and bioavailability standpoint. (Rosoff, supra and Idson supra). Mineral-oil base laxatives, oil-soluble vitamins and high fat nutritive preparations are among the materials that have commonly been administered orally as o/w emulsions.

Liposomes fall into two broad classes. Cationic liposomes are positively charged liposomes, which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147:980-985).

Liposomes, which are pH-sensitive or negatively charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19:269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholiqid and/or phosphatidylcholine and/or cholesterol.

In the majority of aggressive tumorigenic prostate cancer cells, the transcription factor Egr-1 is over-expressed. We provide new insights of Egr-1 involvement in proliferation and survival of TRAMP C2 prostate cancer cells, which express a high constitutive level of Egr-1 protein by the identification of several new target genes controlling growth, cell cycle progression and apoptosis such as cyclin D2, P19ink4d and Fas. Egr-1 regulation of these genes, identified by Affymetrix microarray, was confirmed by real-time PCR, immunoblot and chromatin immunoprecipitation assays. Furthermore we also showed that Egr-1 is responsible for cyclin D2 over-expression in tumorigenic DU145 human prostate cells. The regulation of these genes by Egr-1 was demonstrated using Egr-1 antisense oligonucleotides that further implicated Egr-1 in resistance to apoptotic signals. One mechanism was illustrated by the ability of Egr-1 to inhibit. CD95 (Fas/Apo) expression, leading to insensitivity to Fas L. The results provide a mechanistic basis for the oncogenic role of Egr-1 in TRAMP C2 prostate cancer cells.

### EXAMPLES

### Example 1

### Cell Lines and Culture

TRAMP-C1, TRAMP-C2 and TRAMP-C3 (Foster et al. Cancer Res. 57:3325-3330, 1997) were grown in RPMI-1640 Medium (Invitrogen, Carlsbad, Calif.), supplemented with 5% Fetal Bovine Serum, 2 mM L-glutamine, 100 U/ml penicillin and 100 μg/ml streptomycin (all from Irvine Scientific, Santa Ana, Calif.). DU145 (ATCC HTB-81), C57B1/6, 267B1 and HT1080-E9 cell lines (Huang et al. Cancer Res. 55:5054-5062, 1995) were grown in Dulbecco's Modified Eagle's Medium supplemented with 10% Fetal Bovine Serum, 2 mM L-glutamine, 100 U/ml penicillin and 100 μg/ml streptomycin. All cell lines were maintained in a humidified incubator at 37° C. and 6% CO₂.

### Example 2

### Synthesis of Oligonucleotides

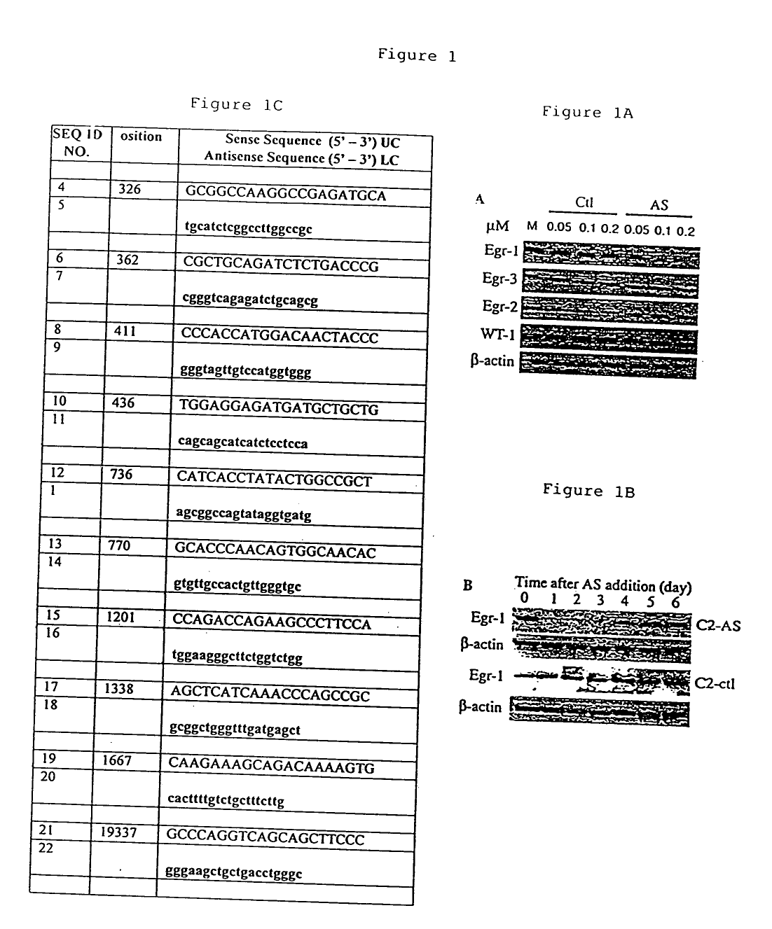

Ten trial antisense oligonucleotides were prepared by Trilink Inc. (San Diego, Calif.) with all phosphorothioate backbone chemistry. They were provided after sequence verification and double HPLC purification. Dry oligonucleotides were resuspended as 10 μM stock solutions in pure sterile water and frozen as aliquots. Position within the mouse gene (ATG=295, accession number NM007913) of each antisense oligonucleotide is shown in (FIG. 1C).
EC (scramble) 5′-TTC TTG CAT CTG TCA-3′ (SEQ ID NO.: 2) and
(mismatch) 5′-AGC GGA CAC TCT AGG CGA TG-3′(SEQ ID NO.: 3).

### Example 3

### Transfection

Transfection of TRAMP-C cells: cells were seeded at a density of 2×10⁵ cells/well in 6-well tissue culture plates the day before transfection in order to achieve 70-80% confluence. Transfection was performed using Lipofectamine. Plus® Reagent (Invitrogen, Carlsbad, Calif.) following the instructions provided by the manufacturer, in a final volume of 1 ml RPMI medium without additives, for 3 h at 37° C. Cells were washed once and maintained in complete medium until the experiment.

Transfection of HT1080-E9 cells: cells were plated at a density of 10⁵ cells/well of a 12-well tissue culture plate the day before transfection. Transfection was performed using Lipofectin® reagent (Invitrogen, Carlsbad, Calif.) following instructions in a final volume of 0.5 ml. After 15 h, cells were washed and maintained in complete medium.

Alternatively, cells were seeded into 35 mm dishes at a density of 100,000 cells per well one day before transfection. The transfection was performed as described by the manufacturer with the GenePorter reagent (16 μl) (Gene Therapy Systems, INC, San Diego, Calif.) and 0.1 μM of antisense oligonucleotide.

### Example 4

### Proliferation Assay

One day before transfection the cells were seeded in duplicate into 35 mm dishes. At day 0 cells were transfected as described above. Four hours later the cells were harvested for counting and for protein and total mRNA extraction. This procedure was repeated each day after transfection according to a time course from day 0 to day 6. To determine cell numbers following transfection, cells were washed twice with PBS, digested by trypsin-EDTA, resuspended in 1.0 ml of 10% serum-containing medium and transferred to a suspension vial in a final volume of 10 ml PBS. Cells were counted using a COULTER™ Multisizer II instrument (Beckman Coulter Inc., Hialeah, Fla.) gated for the appropriate cell size and corrected for particulate debris. Each experiment was performed in duplicates and each vial was counted at least twice. Initial cell numbers were checked by counting after transfection (t=3 h).

### Example 5

### Growth of Cells in Soft Agar

TRAMP-C cells were transfected with control or antisense oligonucleotide (0.1 μM). The day after, cells were trypsinized and seeded in 0.35% (w/v) microbiology grade agarose (Fisher Scientific, Pittsburgh, Pa.) prepared in complete medium. This top layer was poured onto a first layer of sterile 0.5% (w/v) agarose prepared in complete medium that had been allowed to solidify in 6-well plates. After two weeks of incubation at 37° C. in 5% CO₂, colonies were stained with 0.5 mg/ml of nitro-blue tetrazolium (Sigma Aldrich Inc., Milwaukee, Wis.). After 24 h the plates were photographed and grown colonies were counted.

### Example 6

### Cell Death Measurement

The day after transfection, the cells were ultraviolet-C (UVC) irradiated (40 J/m²) in a: Stratalinker (Stratagene, La Jolla, Calif.) or treated with 100 ng/ml of Fas L recombinant protein (Oncogene Research Products, Darmstadt, Germany). One or two days after UVC irradiation or 9 h and 18 h after Fas L treatment, detached and trypsinized cells were pooled and incubated with 0.2% trypan blue to determine the percentage of dead cells.

### Example 7

### Colony Formation Assay

TRAMP C2 cells were transfected as described above. After 16 h the cells were counted and seeded into 6 well plates (200 cells/well) in RPMI medium with 0.1 μM of antisense oligonucleotide. After 8 days incubation at 37° C., the colonies were stained with 2% crystal violet dried and used for photography and colony counting.

### Example 8

### Flow Cytometry Analysis of Cell Cycle

TRAMP-C2 cells were transfected with control or antisense oligonucleotides (0.2 μM). Forty-eight hours later cells were treated with trypsin-EDTA, washed, resuspended in medium, counted, and then fixed in 70% ethanol at 4° C. for 2 h. The cells were washed and resuspended at a concentration of 10⁶ cells/0.5 ml in PBS containing 0.1% Triton X-100, 50 μg/ml DNase-free RNase A, and 50 μg/ml propidium iodine. They were incubated in the dark for 30 minutes at room temperature. The red fluorescence of single events was recorded using an argon ion laser at 488 nm excitation wavelength (FACS Calibur flow cytometer, Becton Dickinson Corp., San Jose, Calif.). Cell Quest™ Software was used for cell cycle histogram determination and data analysis.

### Example 9

### Western Blot Analysis of Protein Expression

Cells were chilled on ice and washed twice with ice-cold Phosphate Buffered saline (PBS: 43‘mM ’ K₂HPO₄; 9 mM Na₂HPO₄; 120 mM NaCl; pH 7.4). They were solubilized on ice in lysis buffer containing 50 mM Hepes pH 7.5; 150 mM NaCl; 100 mM NaF; 10 mM EDTA, 10 mM Na₄P₂O₇; 1% (v/v) Triton X-100; 0.5% Deoxycholic acid, 0.1% SDS and a Protease Inhibitor Cocktail (Sigma Aldrich Inc., St Louis, Mo.). Lysates were then clarified by centrifugation at 13,000×g for 10 min at 4° C. Protein concentration was determined using the BCA™ protein assay reagent (Pierce, Rockford, Ill.). Cleared lysates were resuspended in Sample buffer containing 70 mM Tris-HCl; 10% (v/v) glycerol; 2% (w/v) SDS; 0.01% (w/v) Bromophenol Blue, 1.5% (v/v) 2-mercaptoethanol. Samples were subjected to electrophoresis on a 10% acrylamide gel and transferred to Immobilon-P® membranes (Millipore, Bedford, Mass.) using standard procedures. Membranes were blocked in saline buffer (25 mM Tris-HCl pH 7.4; 140 mM NaCl; 0.1% (v/v) Tween-20) containing 5% (w/v) non-fat milk for 2 h at 22° C. before addition of the antibodies for an overnight incubation at 4° C. Several washes were performed in saline buffer and peroxydase-conjugated antibodies against mouse or rabbit immunoglobulins (Amersham Biosciences, Piscataway, N.J.) were added at a dilution of 1/6000 for 45 min at 22° C. After washing, the membranes were soaked in Western Blotting Luminol Reagent™ (Santa Cruz Biotechnology Inc., Santa Cruz, Calif.) followed by autoradiography. When appropriate, membranes were stripped using Restore™ Stripping Buffer (Pierce, Rockford, Ill.) for 15 min at 22° C. and reprobed with the indicated antibodies.

Proteins were blocked and reacted with antibodies to Egr-1 (C19, Santa Cruz Biotechnology, Inc, Santa Cruz, Calif.), mouse and human Cyclin D2 (sc-593 and sc-181, Santa Cruz Biotechnology, Inc, Santa Cruz, Calif.), p19^{ink4d} (sc-1063, Santa Cruz Biotechnology, Inc, Santa Cruz, Calif.) or CD95 (Anti-mouse Fas/TNFRSF6 (CD95) Antibody, R&D systems, Inc, Minneapolis, Minn.).

### Example 10

### Antibodies

Antibodies to Egr-1 (sc-189), Egr-2 (sc-190) and Egr-3 (sc-191) were purchased from Santa Cruz Biotechnology Inc. (Santa Cruz, Calif.). They were used at a concentration of 0.07 μg/ml and 0.1 μg/ml, respectively (in a total volume of 12 ml). Antibodies to β-actin (clone AC-15) were from Sigma Aldrich Inc. (Saint-Louis, Mo.) and were used at a concentration of 0.22 μg/ml in a total volume of 12 ml.

### Example 11

### Antisense Oligodeoxynucleotide Efficiently Inhibits Egr-1 Expression

To examine the functional significance of Egr-1 over-expression in prostate cancer cells, expression was inhibited using an antisense oligonucleotide (AS) in TRAMP C2 prostate cancer cells. To assess the efficiency and the specificity of AS, we performed western blot analyses of the protein expression of Egr-1 and other Egr family members, Egr-2, Egr-3 and wt1, 24 h after transfection of the antisense and control oligonucleotides (FIG. 1A). As seen in FIG. 1A, the antisense oligonucleotide strongly decreased Egr-1 expression, while there was no effect on Egr-2 and wt1 expression. Egr-3 seems to be slightly increased when Egr-1 was inhibited. In contrast, the control oligonucleotide (ctl) did not alter the protein expression pattern of the cells. These results demonstrated that a 24 h treatment with a low concentration, 0.1 μM, of the AS oligonucleotide efficiently and specifically inhibited Egr-1 expression. To examine the time course of Egr-1 inhibition in C2 cells, proteins were extracted each day for 6 days following AS transfection of antisense and control oligonucleotide-treated cells. Egr-1 expression in the presence of AS was undetectable from day 1 to day 3, became detectable on day 4, and was fully restored on day 5 to day 6 (FIG. 1B, top panel). As expected, the use of the control oligonucleotide (ctl) did not change Egr-1 expression level (FIG. 1B, bottom panel). These results show that AS is stable enough over 3 days to allow almost complete and specific inhibition of Egr-1 expression for a prolonged period following a single treatment.

### Example 12

### Egr-1 Contributes to the Control of Cellular Proliferation

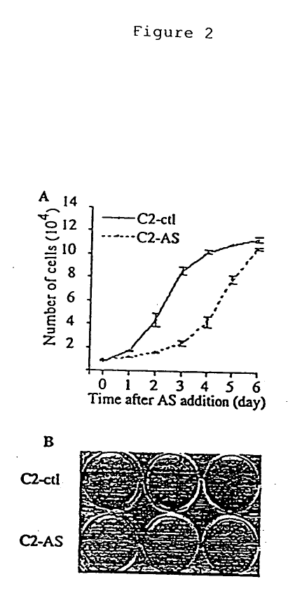

To determine the involvement of Egr-1 in the proliferation rate of TRAMP C2 cells the growth of the cells in which Egr-1 expression was inhibited by AS oligonucleotide (C2-AS), was compared to the control corresponding to TRAMP C2 cells transfected with control oligonucleotide (C2-ctl). Briefly the cells were transfected at day 0 with either AS or ctl and the proliferation rates were directly assessed each day until day 6 by cell counting (FIG. 2A). As seen in FIG. 2A, the proliferation rate of C2-AS cells was strongly reduced during the first three days after transfection and started to rise again on day four. Between day 4 and 5 the slope of the proliferation curve was approximately equal to the slope of the control (C2-ctl cells) indicating that the cells recovered their expected proliferation rate (FIG. 2A). The proliferation time course was well correlated to the pattern of Egr-1 inhibition seen in FIG. 2B. Indeed, as long as Egr-1 expression was inhibited, the proliferation rate of TRAMP C2 cells was markedly reduced and then resumed as soon as Egr-1 expression recovered. In addition, comparison between C2-AS and C2-ctl cells in a colony forming assay showed 74% fewer colonies in C2-AS (average of 32 colonies +/−6 for C2-ctl versus 8.3 colonies +/−3 for C2-AS), suggesting that the tumorigenicity of the cells may decrease when Egr-1 is inhibited (FIG. 2B). Furthermore, cell cycle analysis by FACS, performed at day 2 after transfection, showed fewer cells (about 11% less) in G1 phase of C2-ctl cells than C2-AS cells: (data not shown). The sum of results indicates a role for Egr-1 in the control of growth and cell cycle progression in prostate cancer cells.

### Example 13

### Antisense Oligonucleotides Inhibit TRAMP-C Cell Proliferation

In order to test the role of Egr-1 in growth regulation of TRAMP-C1, TRAMP-C2 and TRAMP-C3, cells were transfected with control or E5 oligonucleotides and initial cell numbers were determined by counting after transfection (t=3 h). Subsequent growth was measured by direct cell counting over a period of 4 days.

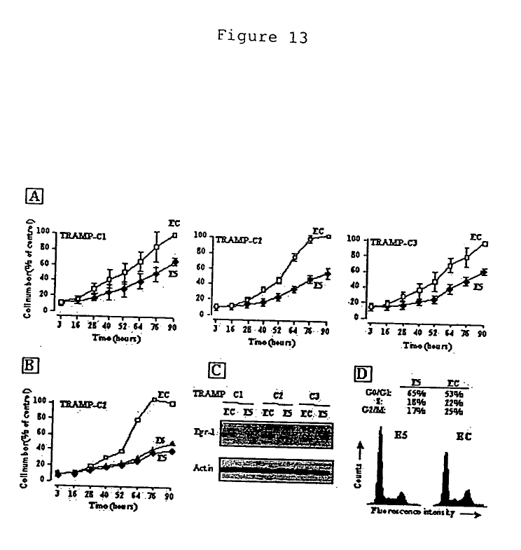

FIG. 13A shows that oligonucleotide E5 inhibited the proliferation of the three cell lines. Inhibition rates were calculated from the integrated growth curves (I %=100(1−Aₐₛ/A_{c})) as determined in three complete replicate experiments for each cell line, where Aₐₛ is the growth curve of antisense-treated cells and A_{c} is the growth curve of control-treated cells. The resulting average values are 47.7%±4.4, 58.3%±5.8, 53.7%±3.3 (n=3) for TRAMP-C1, TRAMP-C2 and TRAMP-C3, respectively, indicating that Egr-1 is required for the normal growth of TRAMP-C cells in vitro.

Indication that inhibition of cell growth was not due to a non-specific toxic effect of the oligonucleotide was provided by the observation that two different oligonucleotides, E5 and E6, gave similar results (FIG. 13B). We monitored antisense-induced inhibition of Egr-1 expression by western analysis: at 28 h following transfection (FIG. 13C), which confirmed that antisense-treated but not control-treated cells exhibited suppressed Egr-1-protein levels. Thus, inhibition of cell growth is likely to be due to inhibition of Egr-1 expression.

Finally, cell cycle analysis by flow cytometry of transfected TRAMP-C2 cells indicated that the antisense oligonucleotide E5 deregulated progression through the cell cycle, with more cells being in the G0/G1 phase when treated with the antisense oligonucleotide compared to control oligonucleotide-treated cells (FIG. 13D). These results seem to indicate that expression of Egr-1 is correlated with accelerated cell cycle.

### Example 14

### Antisense Oligonucleotide E5 Inhibits Colony Formation and Growth in Soft Agar of TRAMP-C1 and TRAMP-C2 Cells

Colony formation and growth in soft agar are hallmarks of transformed cells in vitro. Thus, TRAMP-C1 and TRAMP-C2 that are rapidly tumorigenic when grafted into athymic or autologous C57Bl/6 hosts (TRAMP background) gave positive results in colony formation assay and growth in soft agar. In contrast, TRAMP-C3 cells are poorly tumorigenic and do not form tumors when grafted into these mice (Foster et al., 1997 Cancer Res. 57:3325-3330). Consistently, we found that these cells did not form colonies in vitro and did not grow in soft agar (data not shown).

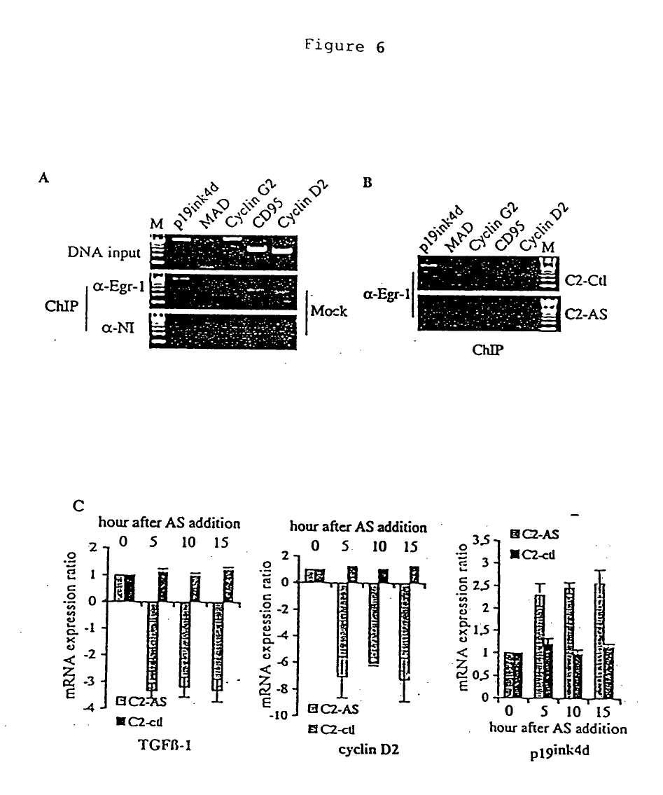

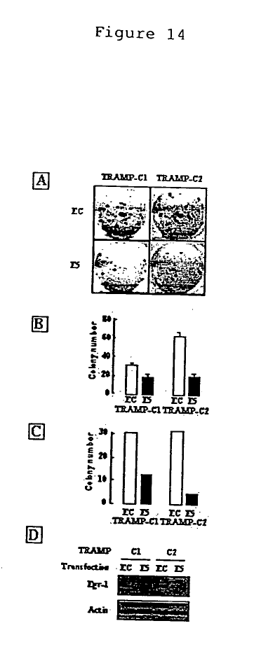

In colony formation assays, TRAMP-C1 and TRAMP-C2 cells were transfected with control or E5 oligonucleotides. They were plated at low density to allow single cell growth for one week. After staining, colony numbers were counted in each culture dish. FIG. 6A shows a picture of a representative dish for each condition and each cell line. FIG. 14B shows the quantification±SE of 3 separate experiments, each performed in triplicate wells.

It is apparent (FIG. 14B) that antisense oligonucleotide E5 treatment inhibited colony formation in both cell lines compared to cells treated with control oligonucleotides. When counted in tissue culture dishes, the colony number of TRAMP-C1 treated with control oligonucleotide was 30.6±2.1, compared to 18.5±2.5 following treatment with E5, which is significant (p<0.005). Colony number of TRAMP-C2 treated with the control oligonucleotide was 61.7±4.2, compared to 20.1±2 when treated with E5, which is also significant (p<0.0005).

### Example 15

### Growth in Soft Agar Reflects Anchorage Independence, Which is a Requisite for Transformation

Cells transfected with control or E5 oligonucleotides were seeded in a top layer of 0.35% agar as described in Example 5. Two weeks later cells were stained with a vital dye to visualize colonies. FIG. 14C shows the colony numbers from two separate experiments. Treatment with antisense oligonucleotide E5 led to a decrease in the number of colonies growing as well as the average size of the colonies in both TRAMP-C1 and TRAMP-C2 cells. For each experiment we monitored inhibition of EGR-1 protein expression by western blot analysis (FIG. 14D).

We conclude that antisense oligonucleotide E5 suppresses two important features of cellular transformation, i.e. colony formation and anchorage-independence for growth.

### Example 16

### Antisense Oligonucleotide E5 Inhibits the Expression of TGF-β

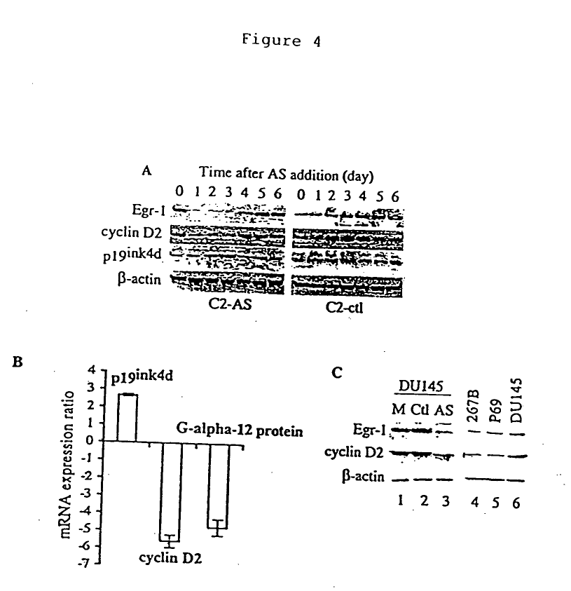

To assess whether the antisense oligonucleotide also inhibits Egr-1 function, we measured levels of mRNA expression for two Egr-1-regulated gene products in parallel with Egr-1 itself. Described targets for Egr-1 include TGF-β1 (Dey et al., 1994 Endogrinology 8:595-602; Liu et al., 1996 PNAS 93:11831-11836), and tumor suppressor PTEN (Virolle et al., 2001 Nature Cell Biol. 3:1124-1128). As shown by RT-PCR experiments (FIG. 4A), antisense oligonucleotide E5 decreased the expression of Egr-1 mRNA in all three TRAMP-C cell lines. Expression of TGF-β1 mRNA was concomitantly down regulated whereas expression of PTEN was not altered. RNA expression levels in TRAMP-C2 cells were also measured by Quantitative Real-Time PCR (Q-PCR). When compared to control, Q-PCR ratios of the E5-treated cells were 0.668, 0.687, and 0.988 for Egr-1, TGF-β1 and PTEN, respectively (a value of 1.0 reflects similar levels in control and antisense-treated cells). These ratios indicate that mRNA levels of Egr-1 and TGF-β1 were concomitantly decreased in E5-treated cells whereas the level of PTEN mRNA was not altered. The latter result was confirmed by examination of PTEN protein levels, which remained unchanged (FIG. 4B). We conclude that the antisense oligonucleotide E5 inhibits Egr-1 transcriptional activity towards TGF-β1. Interestingly, PTEN expression is not regulated by Egr-1 in these cells.

### Example 17

### Antisense Oligonucleotide E5 Decreases Tumor Incidence in TRAMP Mice

PCR-confirmed male TRAMP mice were divided into three treatment groups: saline buffer alone (PBS), mismatch control oligonucleotide, or antisense oligonucleotide E5. The average age of the mice at the start of the treatment was 21.8±0.33 (n=23) when prostate cancer is already developing (Dey et al., 1994 Mol. Endocrinol. 8:595-602). Mice received systemic intraperitoneal injections three times a week with vehicle alone (7 mice), three-base mismatch control oligonucleotide (8 mice) or E5 antisense oligonucleotide (8 mice), at a dose of 25 mg/kg. Animals were sacrificed when showing signs of illness or when tumors became palpable at which time necropsies were carried out. In order to have an age-matched group; random mice of the same generation in the other groups were sacrificed together with tumor-bearing animals. Thus, the average age at sacrifice for the treated animals was 31±0.7 weeks for all three groups (31.9±0.9; 32.4±0.9; 31.1±1.7 in the saline buffer, control and antisense oligonucleotide group, respectively). The average length of treatment was 69±5 days (˜10 weeks) for all groups.

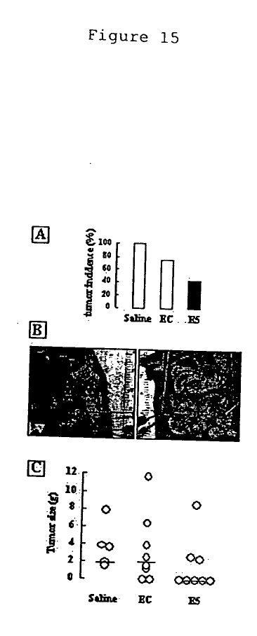

FIG. 15A shows that the incidence of tumor in antisense-treated mice was lower than that of control mice. All seven mice of the saline buffer group and six out of eight mice in the control oligonucleotide-treated group developed tumors characterized by grossly enlarged prostate glands with extensive and typically bilateral involvement of the vesicular glands. In contrast, only three out of eight mice in the ES antisense-treated group developed tumors.

FIG. 15B shows a 32 week-old TRAMP mouse treated with antisense oligonucleotide E5, with normal prostate and seminal vesicle size (right panel) whereas a 27 week-old TRAMP mouse treated with control oligonucleotide exhibited large, obstructive prostate tumor (left panel).

FIG. 15C displays the tumor size for each animal, with the medians indicated as horizontal lines (Median=2, 2.05 and 0 g for saline buffer, control and E5 oligonucleotides, respectively). Average weight at death was 31.9±0.6 g; in which mice with tumor weighed 32.8±0.4 g and mice without tumor weighed 28.3±1. The average weight of tumors was 4.1±0.5 g for all tumor-bearing mice.

The differences between groups were analyzed by the Fisher Exact Test, which displays how different treatments have produced different outcomes. Its null hypothesis is that treatments do not affect outcomes—that the two are independent.

The difference in the incidence of tumors between the oligonucleotide control-treated group and the saline buffer group is not significant (6/8 vs. 7/7; p=0.467). In contrast, when the incidence of tumor for the antisense Egr-1 treated group is compared to the saline buffer group (3/8 vs. 7/7), the difference is significant, p=0.026. When results are analyzed taking all three treatment groups together, the outcome is also significant, with p=0.037 (7/7 vs. 6/8 vs. 3/8). Thus, the control oligonucleotide has no effect on the incidence of tumor whereas E5 antisense oligonucleotide significantly delays tumor incidence in TRAMP mice.

### Example 18

### Egr-1 Desensitizes Cancer Cells to Fas L Induced Apoptosis

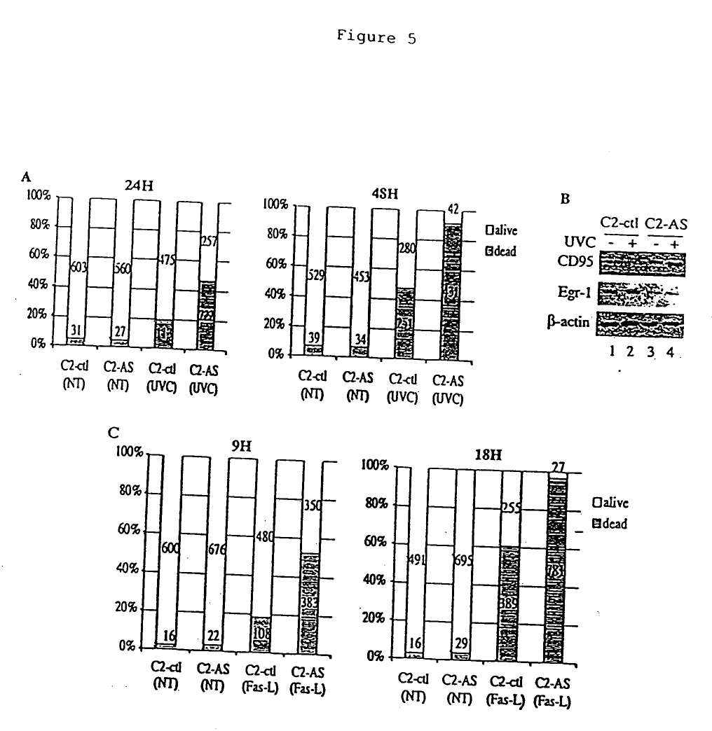

The role Egr-1 may play in promoting prostate cancer by affecting prostate cell survival (Haung et al. 1997 Int. J. Cancer 72:102-109) or apoptosis (Virolle et. al. 2001 Nat. Cell Biol. 3:1124-1128) was determined by UVC irradiating C2-AS and C2-ctl cells. Dead cells were counted by trypan blue staining at 24 and 48 h following irradiation. While less than 20% of the C2-ctl cells were dead 24 h following irradiation, almost 50% of C2-AS cells were dead (FIG. 5A). Furthermore, at 48 h following irradiation, less than 50% of control cells versus 95% for C2-AS cells had died (FIG. 5A). These differences demonstrate a critical role for Egr-1 in response to stress. Indeed, endogenous expression of Egr-1 is not only required for full proliferation of C2 cells but also to decrease sensitivity to radiation, a widely observed phenomenon of human prostate cancer cells (Howell 2000 Mol Urol. 4:225-229).

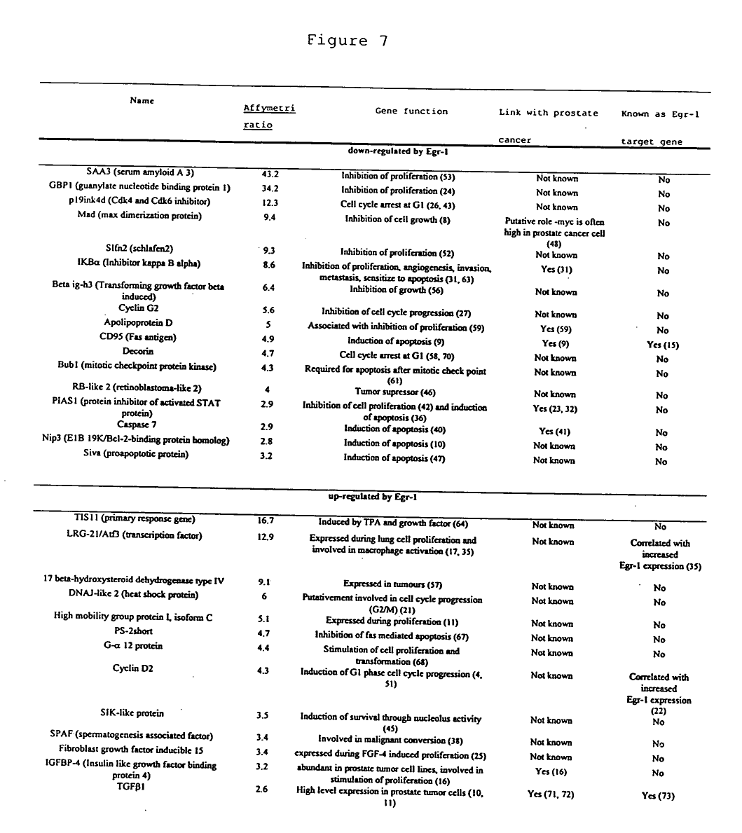

Affymetrix analysis (FIG. 7) of this study revealed several genes that are down regulated by Egr-1, such as caspase 7 (Bowen et al. 1999 Cell Death Differ. 6:394-401, Marcelli et al., supra), Bcl-2-binding protein homolog Nip3 (Chen et al. 1999 J. Biol. Chem., 274:7-10) and CD95 (Fas antigen) (Chatterjee, supra), a gene widely involved in apoptosis pathways. CD95, a member of tumor necrosis factor receptor family, is referred as “death receptor” because of its ability to transduce death signals. On the other hand, the gene PS-2short (unregulated by Egr-1, see FIG. 7) is involved in inhibition of Fas mediated apoptosis (Vito et al. 1996 Science 271:512-525 and Vito et al. 1996 J. Biol. Chem. 271:31025-31028), therefore supporting a role for Egr-1 as anti-apoptotic agent in prostate cancer cells.

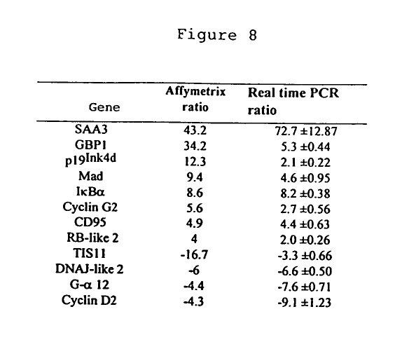

Egr-1 regulation of CD95, although confirmed at the mRNA level by real time PCR (FIG. 8), was also tested for protein expression in C2-ctl and C2-AS cells treated or not by UVC irradiation. In C2-ctl cells, UVC treatment led to a significant increase of Egr-1 expression, which was strongly inhibited by AS (FIG. 5B, C2-AS). CD95 expression appeared to be undetectable in C2-ctl-treated cells but was clearly expressed in C2-AS-treated cells (FIG. 5B). After UVC treatment CD95 expression was strongly increased in C2-AS while it was only slightly expressed in C2-ctl (FIG. 5B). These results confirm at the protein level the efficient inhibition of CD95 expression by Egr-1. This mechanism of repression is all the more relevant since it is still effective even after a strong stress stimulus.

In order to assess whether this difference in basal CD95 expression could be reflected as responses to Fas L mediated apoptosis, we treated C2-ctl and C2-AS cells for 9 and 18 h with Fas L, and counted the percentage of dead cells by Trypan Blue staining. As expected from CD95 protein expression profile (FIG. 5B), C2-AS were more sensitive to Fas L mediated apoptosis. Indeed, at 9 h after treatment, 52% of cells were dead in C2-AS versus 18.5% in C2-ctl cell cultures (FIG. 5C). This difference in the resistance to cell death between C2-AS and C2-ctl cells, although lower, was still present after 18 h treatment, with 96% of dead cells compared to 60%, respectively (FIG. 5C). Therefore high constitutive Egr-1 expression delays apoptosis of prostate cancer cells mediated by Fas L, in part by down regulating CD95 expression. The significance of the CD95 signaling pathway in prostate apoptosis has also been demonstrated in the normal rat prostate following castration (de la Taille et al. 1999 Prostate 40:89-96). In addition, further studies have demonstrated the involvement of CD95 in sensitizing prostate cancer cells to undergo apoptosis after chemotherapeutic agent or irradiation treatments (Costa-Pereira and Cotter 1999 Br. J. Cancer 80:371-378 and Kimura and Gelmann 2000 J. Biol. Chem. 275:8610-8617). These results illustrate well a “desensitizer role” of Egr-1 in the cell death response and suggest that sensitization to Fas mediated apoptosis by the inhibition of Egr-1 expression could become an attractive therapeutic mechanism. Furthermore this experiment presents corroborating evidence that the modification of gene expression by Egr-1 is a major player in the pathological responses of prostate cancer cells.

### Example 19

### p19^{ink4d}, Mad, CD95 and Cyclin D2 are Directly Transcriptionally Regulated by Egr-1

Gene chip and real time PCR technologies are powerful and sensitive enough to accurately evaluate the differential expression between two mRNA populations but do not determine if the regulation by Egr-1 occurs directly or indirectly. Therefore, we performed chromatin cross-linking and immunoprecipitation assays (ChIP) to screen upstream regulatory sequences of five examples of putative Egr-1 target genes indicated by the Affymetrix analysis. For this experiment untransfected, AS and Ctl oligonucleotides transfected C2 cells were used as template. After chromatin cross-linking in living cells, Egr-1 became covalently fixed to its DNA target. These captured target DNA fragments were then recovered by specific Egr-1 immunoprecipitation and purification. Non-immune serum immunoprecipitation was used as the negative control and C2 genomic DNA was used to asses amplification efficiency of each primers pair. Primers were designed to specifically recognize 5′ regulatory sequences of p19^{ink4d}, Mad, CD95, cyclin G2 and cyclin D2, in order to detect their presence in the captured DNA fragments by polymerase chain reaction. 5′ regulatory sequence analysis of each of these genes showed several putative Egr-1 and Sp-1 binding sites. p19^{ink4d}, Mad, CD95 and cyclin D2 yielded an amplified product from untransfected (Mock) (FIG. 6A) and Ctl oligonucleotide transfected template (FIG. 6B), that showed the same migration pattern as the genomic control input while Cyclin G2 was not detected (FIG. 6 A & B). Since no amplification was found for the control non-immune serum template (FIG. 6A) and the AS oligonucleotide transfected template (FIG. 6B), these results indicate that the successfully amplified fragments were bound by Egr-1 in vivo and therefore indicate the direct regulation of p19^{ink4d}, Mad, CD95 and cyclin D2 by Egr-1. Furthermore, to rule out the possibility that these genes could be regulated in consequence of the inhibition of the proliferation we performed a kinetic of regulation at early time in parallel of TGF-β1 a well known Egr-1 target gene (Liu et al. 1999 J. Biol. Chem. 12:4400-4411). Since AS oligonucleotide is efficient at 5 hours after transfection (data not shown), we performed the kinetic analysis at 5 h, 10 h and 15 h. As for TGF-β1, the modulation of cyclin D2 and p19^{ink4d} expression occurred at 5 hours after AS addition corresponding to the onset of Egr-1 efficient inhibition (FIG. 6C). Taken together these results indicate that many of the Egr-1 target genes identified in our study may be regulated directly by Egr-1.

1. A oligonucleotide up to 30 bases in length comprising at least an 8 nucleobase portion of 5′-AGC GGC CAG TAT AGG TGA-3′ (SEQ ID NO.1).

2. A oligonucleotide according to claim 1 wherein said oligonucleotide is an antisense oligonucleotide.

3. A oligonucleotide according to 1 bound to a complimentary RNA forming a double stranded interference RNA.

4. An oligonucleotide according to 1 comprising at least one modified internucleosidyl linkage.

5. An oligonucleotide according to 2 wherein said modified internucleosidyl linkage is at least one phosphorothioate internucleosidyl linkage.

6. An oligonucleotide according to 2 wherein said modified internucleosidyl linkage is at least one methylphosphonate internucleosidyl linkage.

7. An oligonucleotide according to 2 wherein said modified internucleosidyl linkage is at least one phosphodiester internucleosidyl linkage.

8. An oligonucleotide according to 2 wherein said oligonucleotide comprises at least one methylphosphonate and at least one phosphodiester analog.

9. An oligonucleotide according to 2 wherein said oligonucleotide comprises at least one methylphosphonate and at least one phosphorothioate analog.

10. An oligonucleotide according to 2 wherein said oligonucleotide comprises at least one phosphorothioate and at least one phosphodiester analog.

11. An oligonucleotide according to 2 wherein said oligonucleotide comprises at least one methylphosphonate, at least one phosphorothioate and at least one phosphodiester analog.

12. An oligonucleotide according to 1 wherein said oligonucleotide is a chimeric oligonucleotide.

13. An oligonucleotide according to 1 comprising at least one modified sugar moiety.

14. An oligonucleotide according to 1 comprising at least one modified nucleobase moiety.

15. A composition comprising the oligonucleotide according to 1 and a pharmaceutically acceptable carrier.

16. A vector comprising the oligonucleotide sequence according to 1.

17. A plasmid comprising a vector according to 1.

18. A cell comprising an oligonucleotide according to 1.

19. A method for the treatment of cancer cells wherein Egr-1 is over-expressed as a result of the cancer comprising the steps of:
administering an oligonucleotide according to 1 to an animal having said cancer cells in which Egr-1 is over-expressed for a time and until said Egr-1 expression is reduced.

20. A method according to 19 wherein said treatment results in decreased proliferation of said cancer cells.

21. A method according to 19 wherein said treatment results in increased apoptosis of said cancer cells.

22. A method according to 19 wherein said treatment results in reduced expression of transforming growth factor beta-1 or interleukin-6.

23. A method according to 19 wherein said treatment further results in reduced expression of Cyclin D2 and G-alpha-12 in said cancer cells.

24. A method according to 19 wherein said treatment further results in increased expression of Cyclin G2 and p19^{ink4d}.

25. A method according to 19 wherein said cancer cells are prostate cancer cells.

26. A method of interfering with the growth of cancer cells, wherein said cancer cells over-express the Egr-1 gene comprising the steps of;
(a) introducing an oligonucleotide according to 1 to said cancer cells; and
(b) contacting said cancer cells with an amount of at least one chemotherapeutic agent sufficient to kill a portion of said cancer cells whereby said portion of said cancer cells killed is greater than the portion which would have been killed by the same amount of chemotherapeutic agent in the absence of said oligonucleotide.

27. A method according to 26 wherein said cancer cells are prostate cancer cells.

28. A kit comprising a composition according to 15 and a chemotherapeutic agent.

29. A kit comprising a vector according to 16 and a chemotherapeutic agent.

30. A composition comprising an iRNA according to 3 and a pharmaceutically acceptable carrier.

31. A cell comprising an iRNA according to 3.

32. A method for the treatment of cancer cells wherein Egr-1 is over-expressed as a result of the cancer comprising the steps of:
administering an iRNA according to 3 to an animal having said cancer cells in which Egr-1 is over-expressed for a time and until said Egr-1 expression is reduced.

33. A method according to 32 wherein said treatment results in reduced expression of transforming growth factor beta-1 or interleukin-6.

34. A method according to 32 wherein said treatment results in decreased proliferation of said cancer cells.

35. A method according to 32 wherein said treatment results in increased apoptosis of said cancer cells.

36. A method according to 32 wherein said treatment further results in reduced expression of Cyclin D2 and G-alpha-12 in said cancer cells.

37. A method according to 32 wherein said treatment further results in increased expression of Cyclin G2 and p19^{ink4d}.

38. A method according to 32 wherein said cancer cells are prostate cancer cells.

39. A method of interfering with the growth of cancer cells, wherein said cancer cells over-express the Egr-1 gene comprising the steps of;
(c) introducing an iRNA according to 3 to said cancer cells; and
(d) contacting said cancer cells with an amount of at least one chemotherapeutic agent sufficient to kill a portion of said cancer cells whereby said portion of said cancer cells killed is greater than the portion which would have been killed by the same amount of chemotherapeutic agent in the absence of said oligonucleotide.

40. A method according to 39 wherein said cancer cells are prostate cancer cells.

41. A kit comprising a composition according to 30 and a chemotherapeutic agent.
